(19) United States
(12) Patent Application Publication
(10) Pub. No.: US 2013/0190619 A1
(43) Pub. Date: Jul. 25, 2013

(54) Angiography Catheter

(71) Applicant: Ron Nudel, Woodbridge, CT (US)

(72) Inventor: Ron Nudel, Woodbridge, CT (US)

(21) Appl. No.: 13/693,422

(22) Filed: Dec. 4, 2012

Related U.S. Application Data
(60) Provisional application No. 61/567,195, filed on Dec. 6, 2011.

(51) Int. Cl. A61M 25/01

(52) U.S. Cl. CPC A61M 25/0102
USPC 600/434; 600/407

(57) ABSTRACT

A catheter assembly having a relatively stiff proximal portion bonded to a conformable distal portion with a first lumen extending through the proximal portion and through the distal portion. Also provided is a plurality of stylets each having a different preformed shape and each sized for insertion into the first lumen. Each stylet is effective to shape the distal end to a predetermined shape. Alternatively, the stylets may be received in a second lumen formed in a wall of the proximal portion and of the distal portion.

### CROSS REFERENCE TO RELATED APPLICATION(S)

This patent application claims priority to the filing date of U.S. Provisional Patent Application Ser. No. 61/567195, titled “Angiography Catheter,” that was filed on Dec. 6, 2011. The disclosure of U.S. 61/567195 is incorporated by reference herein in its entirety.

### U.S. GOVERNMENT RIGHTS

N.A.

### BACKGROUND

1. Field of the Disclosure

The disclosure relates to a catheter useful for angiography. More particularly, a pre-shaped stylet inserted into the catheter in situ effects changes or modifies the shape of the catheter without requiring a formal catheter exchange.

2. Description of the Related Art

Angiography can require use of multiple catheters if standard sized and shaped catheters do not fit. Each exchange requires additional time and introduces additional risk into the procedure. In difficult cases, an unsuccessful catheter exchange can quickly undo slowly and arduously gained procedural progress.

The need for multiple catheters requires significant storage space. There are often logistical ordering issues because unusual cases can use up unusual amounts of equipment. Also, the per case equipment cost can rise quickly with multiple catheter use. In addition, there are even times when none of the preformed catheters fit sufficiently. Required are, in short, multiple catheters because angiography equipment is not “one-size-fits-all.”

Exemplary is Optitorque™ Coronary Diagnostic Catheters by Terumo Interventional Systems (Somerset, N.J.), that is incorporated by reference herein in its entirety. Disclosed are 19 coronary diagnostic catheters having a length of between 100 cm and 110 cm and different tip shapes. Among the tip shapes known in the technology are Tiger, Jacky, Pigtail, Judkins Left, Judkins Right, Bypass and Amplatz.

In coronary angiography, the radial artery approach, introducing the catheter via the main artery in a wrist, is being more frequently utilized. It has been found that despite greater technical difficulties, the procedure is safer and more comfortable for the patient. The downside is that more catheter exchanges may be required, more time may be needed, and more radiation exposure to the patient and to the operator may occur.

U.S. Pat. No. 4,033,331 discloses a catheter having a deformable distal tip with a preformed shape. A wire inserted in a separate wire lumen may extend almost to the end of the distal tip forming a relatively straight end. Pulling back the wire a first amount causes the distal tip to assume a first curvature and pulling back the wire a second amount causes the distal tip to assume a second curvature. The distal tip curvatures of three conventional catheters is disclosed to be obtained from a single catheter.

U.S. Pat. No. 4,925,445 discloses a catheter guide wire formed from a super elastic metal alloy that resists buckling. The cross-sectional diameter of the wire body is greater than the cross-sectional diameter of the distal end of the wire. Disclosed alloys include Ti—Ni; Cu—Zn; Cu—Zn—X, where X is Be, Si, Al or Ga; and Ni—Al.

U.S. Pat. No. 5, 290,229 discloses a transformable catheter having an inner catheter with a distal end having a pre-formed shape and an outer sheath. Extending the sheath over a portion of the length of the distal end changes the shape of that distal end.

Each of U.S. Pat. Nos. 4,033,331; 4,925,445; and 5,290,299 is incorporated by reference herein in its entirety.

While described herein for coronary angiography, the disclosed embodiments many be utilized in any angiography.

### BRIEF SUMMARY

In accordance with a first embodiment of the disclosure, there is provided a catheter assembly having a relatively stiff proximal portion bonded to a relatively conformable distal portion with a first lumen extending through the proximal portion and at least partially through the distal portion. Also provided is a plurality of stylets or shaping ribbons each having a different preformed shape and each sized for insertion into the first lumen. Each of the plurality of stylets or shaping ribbons is effective to shape the distal end to the preformed shape.

In accordance with a second embodiment of the disclosure, there is provided a method to use a catheter assembly. This method includes inserting a catheter having a relatively stiff proximal portion bonded to a relatively conformable distal portion with a first lumen extending through the proximal portion and at least partially through the distal portion into a body channel. A first stylet or shaping ribbon having a first preformed shape is inserted into the first lumen whereby the distal end assumes the first preformed shape. Then the first stylet or shaping ribbon is removed and a second stylet or shaping ribbon having a second preformed shape, that is different from the first preformed shape, is inserted into the first lumen whereby the distal end assumes the second preformed shape.

Among the features and advantages of the embodiments disclosed here in are that a shapeable angiography catheter can be transformed in-situ to entirely different shapes or can be modified slightly without a formal catheter exchange. Stylets used to shape the shapeable angiography catheter can be pre-shaped and can be further modified as needed for each situation. The stylets or shaping ribbons take less space to store than an equivalent number of pre-shaped catheters. The stylets or shaping ribbons enable a limited number of catheters to be conformable to limitless shapes and fewer catheter exchanges mean quicker procedure time, less radiation, and less risk to the patient.

The details of one or more embodiments of the invention are set forth in the accompanying drawings and the description below. Other features, objects and advantages of the invention will be apparent from the description and drawings, and from the claims.

### BRIEF DESCRIPTION OF THE DRAWINGS

FIG. 1A illustrates a shapeable angiography catheter for use with the stylets and shaping ribbons described herein.

FIG. 1B is an enlarged view of a distal tip section of the angiography catheter illustrated in FIG. 1A.

FIG. 2A a conformable section of the catheter of FIG. 1A prior to shaping.

FIG. 2B illustrates the conformable section shown in FIG. 2A subsequent to shaping.

FIG. 3A illustrates in cross-sectional representation a first embodiment of a lumen intended to receive a stylet or shaping ribbon as disclosed herein.

FIG. 3B illustrates in cross-sectional representation a second embodiment of a lumen intended to receive a stylet or shaping ribbon as disclosed herein.

FIG. 3C illustrates in cross-sectional representation a third embodiment of a lumen intended to receive a stylet or shaping ribbon as disclosed herein.

FIG. 4A is a perspective view of a first insertion site effective to introduce a stylet or shaping ribbon into the shapeable angiography catheter of FIG. 1.

FIG. 4B is a longitudinal cross-sectional view of the first insertion site of FIG. 4A.

FIG. 4C is a radial cross-sectional view of the first insertion site of FIG. 4A.

FIG. 5A is a perspective view of a second ingress section effective to introduce a stylet or shaping ribbon into the shapeable angiography catheter of FIG. 1.

FIG. 5B is a longitudinal cross-sectional view of the first insertion site of FIG. 5A.

FIG. 5C is a radial cross-sectional view of the first insertion site of FIG. 5A.

FIG. 6A illustrates how the conformable section of the catheter of FIG. 1 may be changed in situ by changing stylets or shaping ribbons to a straight stylet.

FIG. 6B illustrates how the conformable section of the catheter of FIG. 1 may be changed in situ by changing stylets or shaping ribbons to a hooked L shaped stylet.

FIG. 6C illustrates how the conformable section of the catheter of FIG. 1 may be changed in situ by changing stylets or shaping ribbons to a pigtail shaped stylet.

FIG. 7A illustrates a hemodynamic catheter being shaped by the stylet or shaping wire described herein.

FIG. 7B is a broken partial view of the hemodynamic catheter of FIG. 7A.

FIG. 8A illustrates an in situ change of a catheter distal end between having a Jacky shape.

FIG. 8B illustrates the catheter distal end of FIG. 8A having been changed to a Sarah shape.

FIG. 9A illustrates an in situ change of a catheter distal end between having a Jacky shape.

FIG. 9B illustrates the catheter distal end of FIG. 9A having been changed to a pigtail shape.

FIG. 10A illustrates a catheter distal end shaped for angiography.

FIG. 10B illustrates the catheter distal end of FIG. 10A shaped for percutaneous coronary intervention (PCI).

Like reference numbers and designations in the various drawings indicated like elements.

### DETAILED DESCRIPTION

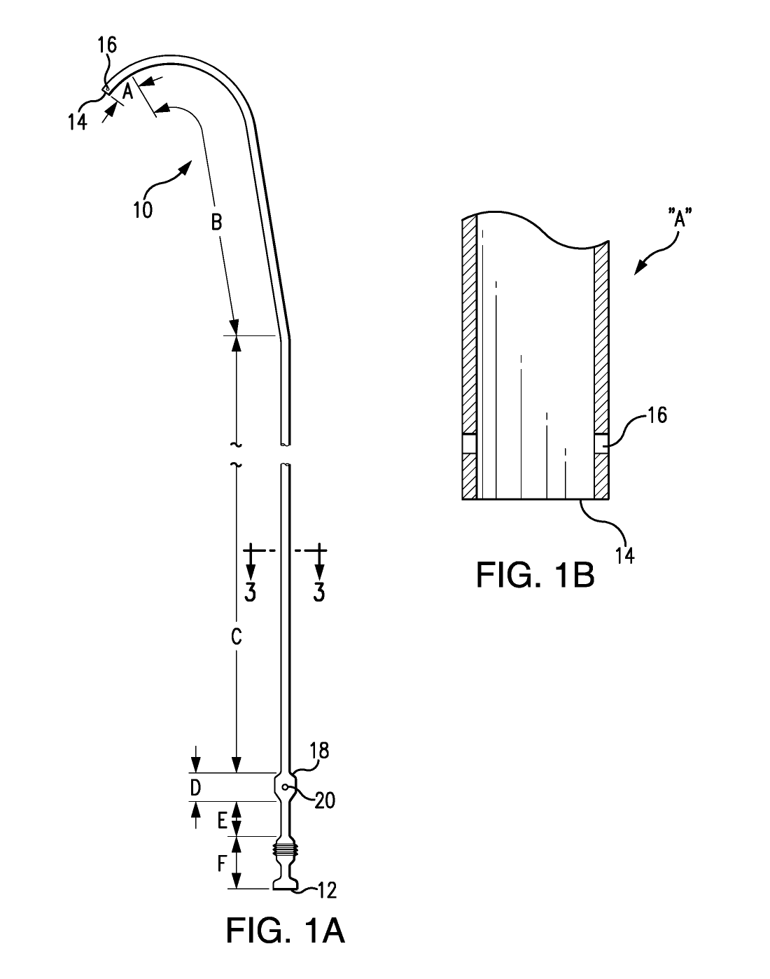

A shapeable angiography catheter 10 is illustrated in FIG. 1A. It is of standard angiography length, nominally either 100 cm or 110 cm in length and a diameter of 5 French or 6 French (1.67 mm or 2 mm) or slightly larger, and may be divided into sections, A-F, with each section seamlessly leading to the next. The shapeable angiography catheter 10 is tubular with a proximal opening 12 and a distal opening 14 making it appropriate for pressure measurement and contrast injection for angiography. With reference to FIG. 1B., section A is a relatively short tip that contains an end hole at distal opening 14. For some applications, section A also includes two side holes 16. Section A is formed from a relatively rigid material. As used herein, “relatively rigid” means that the section does not change shape when contacted by a stylet or shaping ribbon. “Conformable” means that the section does change shape when contacted by the stylet or shaping ribbon.

Referring back to FIG. 1A, section B is tubular and formed from a conformable material. Section C is fused to section B and is formed from a relatively rigid standard angiography catheter material. Section D includes a rubberized insertion site 18 having a stylet insertion site 20 filled with a self-sealing polymer. This section is similar to a small medicine bottle holding a liquid that can be removed using a syringe and a needle pushed through a rubberized stopper. The stopper is self sealing. Similar material can be used for this rubberized section. Sections E and F form a standard proximal hub that attaches to a manifold.

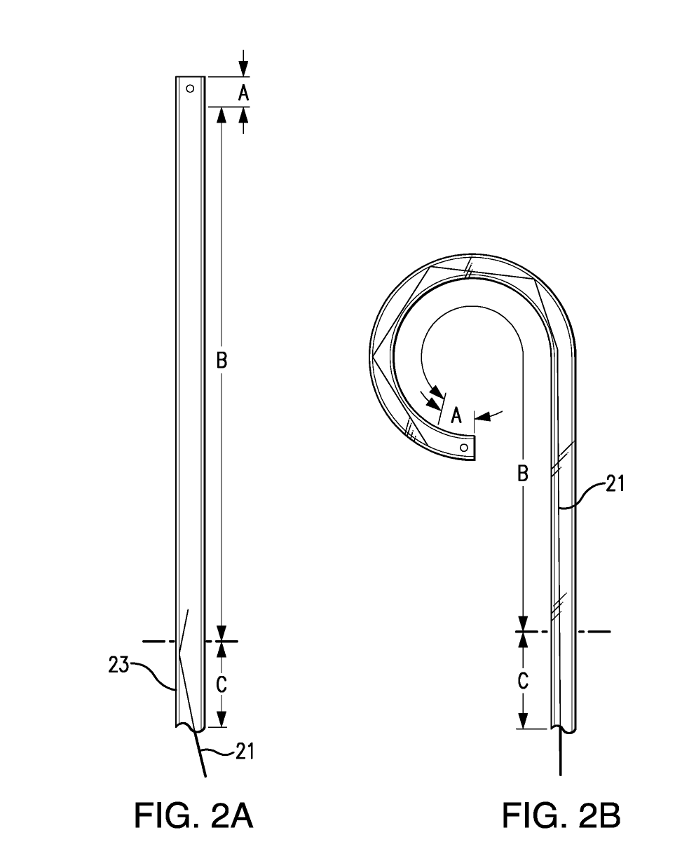

FIGS. 2A and 2B illustrate how conformable section B changes shape in situ by insertion of a stylet or shaping ribbon 21. Both a stylet and a shaping ribbon are relatively elastic materials having a predetermined shape. While the stylet or shaping ribbon may deform from that shape in rigid section C, it returns to the predetermined shape on entering the conformable section B. The word “stylet” is used herein to convey any extended length object capable of achieving this objective. In FIG. 2A, stylet 21 is deformed from its predetermined shape by contact with sidewall 23 of section C. As shown in FIG. 2B, when inserted into conformable section B, the stylet reverts to the predetermined shape deforming section B to a desired configuration, such as approximately 270° curvature in FIG. 2B.

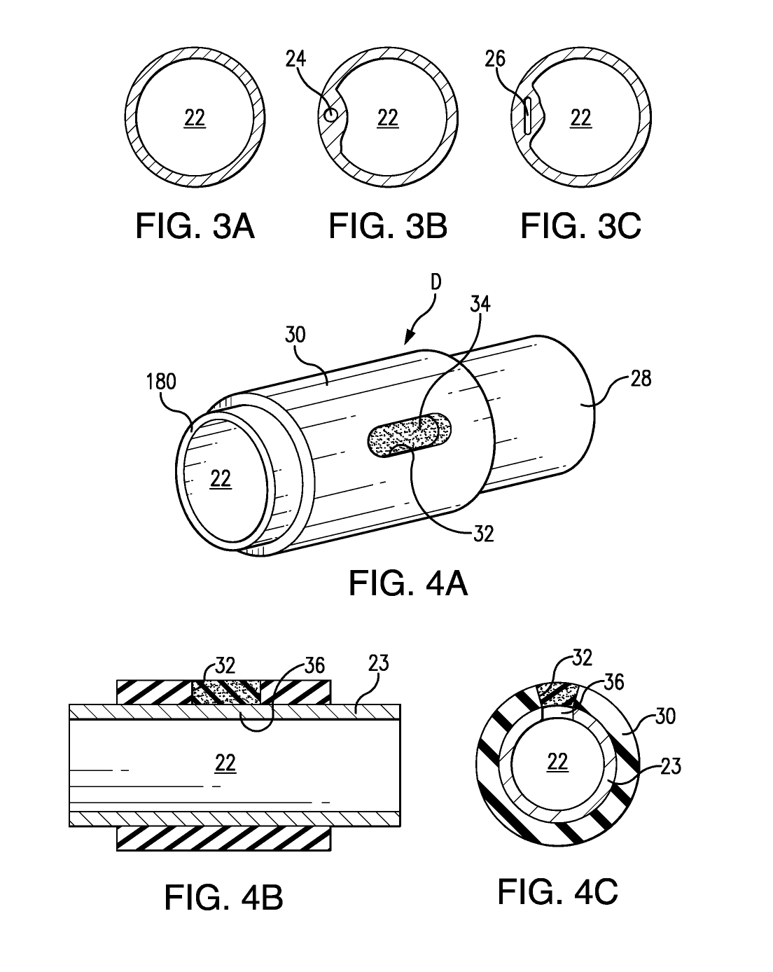

FIGS. 3A-3C are cross sectional views of the catheter taken through section 3-3 of FIG. 1A. A first embodiment, as illustrated in FIG. 3A, includes a single lumen 22 where the stylet is inserted into the same lumen used for introducing a dye for angiography. In a second embodiment, as illustrated in FIG. 3B, there are separate lumens for the stylet 24 and for the angiography dye 22. In a third embodiment, as illustrated in FIG. 3C, there are separate lumens for a shaping ribbon 26 and for the angiography dye 22. Another alternative is to start with a separate lumen 24, 26 through section C and then transition to a single lumen 22 in section B. A stylet may be extended into section B, but does not enter section A. The stylet has a nominal length equal to the length of section B+the length of Section C+the portion of Section D extending from distal end 18 to stylet insertion site 20.

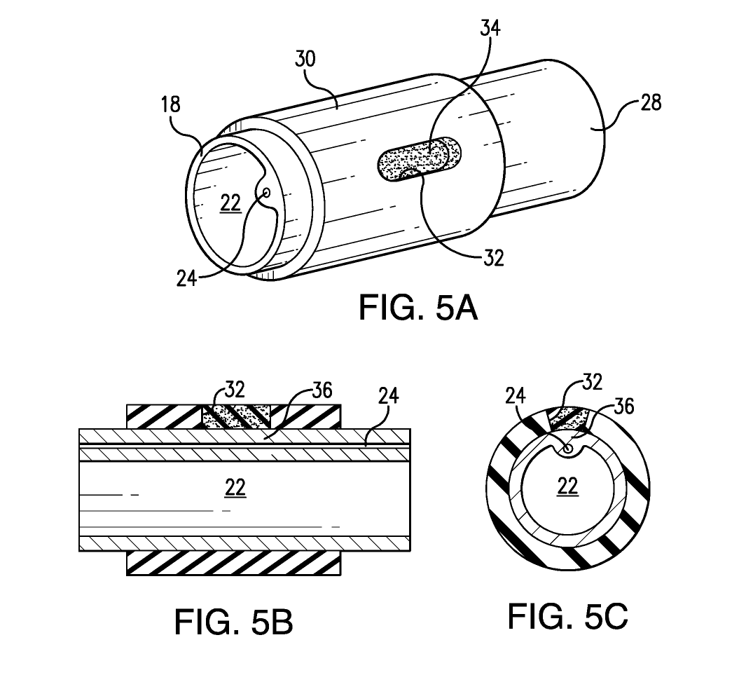

FIGS. 4A-4C and 5A-5C give detail about the rubberized stylet insertion section D. FIG. 4A illustrates section D for a first embodiment where the stylet is inserted in the same lumen 22 as used for angiography dye. Section D has a distal end 180 that is joined to section C and a proximal end 28 that is joined to section E. A polymer jacket 30, typically formed from a rubberized material, circumscribes a mid-portion of section D. A first aperture 32 formed within the polymer jacket 30 is filled with a self-healing rubberized material. The viscous gel 34 is effective to maintain a tight seal around a stylet when inserted and to seal the first aperture 32 when a stylet is not inserted.

FIG. 4B is a longitudinal cross-section view of section D and FIG. 4C is a radial cross-sectional view of section D showing a second aperture 36 extending through catheter sidewall 23 in alignment with the first aperture 32. This provides a passage for the stylet or shaping ribbon to lumen 22.

FIG. 5A illustrates section D for a second embodiment where the stylet is inserted into a lumen 24 that is separate from the lumen 22 used for angiography. As described above, section D has a distal end 18 that is joined to section C and a proximal end 28 that is joined to section E. A polymer jacket 30, typically formed from a rubberized material, circumscribes a mid-portion of section D. A first aperture 32 formed within the polymer jacket 30 is filled with a self-healing rubberized material. The viscous gel 34 is effective to maintain a tight seal around a stylet or shaping ribbon when inserted and to seal the first aperture 32 when a stylet or shaping ribbon is not inserted.

FIG. 5B is a longitudinal cross-section view of section D and FIG. 5C is a radial cross-sectional view of section D showing the second aperture 36 extending through section D in alignment with the first aperture 32. This provides a passage for the stylet to stylet lumen 24.

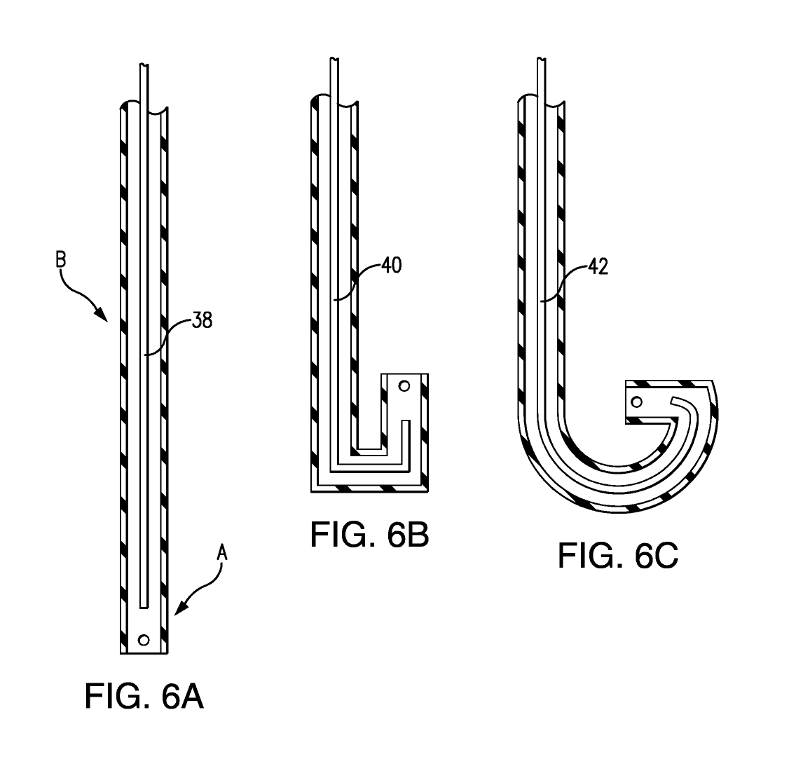

FIGS. 6A, 6B and 6C illustrate how different stylets change the shape of the conformable section B. These shape changes may be conducted in situ by removing a first shaped stylet or shaping ribbon and replacing with a second, different shaped, stylet or shaping ribbon. FIG. 6A illustrates a straight stylet 38 extending the length of conformable section B, but terminating prior to distal end A. Such a straight stylet is useful for inserting a catheter for coronary/vascular angiography through the vascular system until approaching the heart. To traverse sections of the heart, or other non-linear passageways, straight stylet 38 is used to position distal end A adjacent a location where a turn is required. The straight stylet is then removed and replace with a stylet having a different shape, such as a hooked L shape 40 (L4 stylet) or a pigtail shape 42. Multiple progressions of pre-shaped stylets or shapeable stylets may be utilized in a single procedure. It is within the scope of the disclosure to include a kit having a catheter and a plurality of different sized and shaped stylets to meet a physician's requirements.

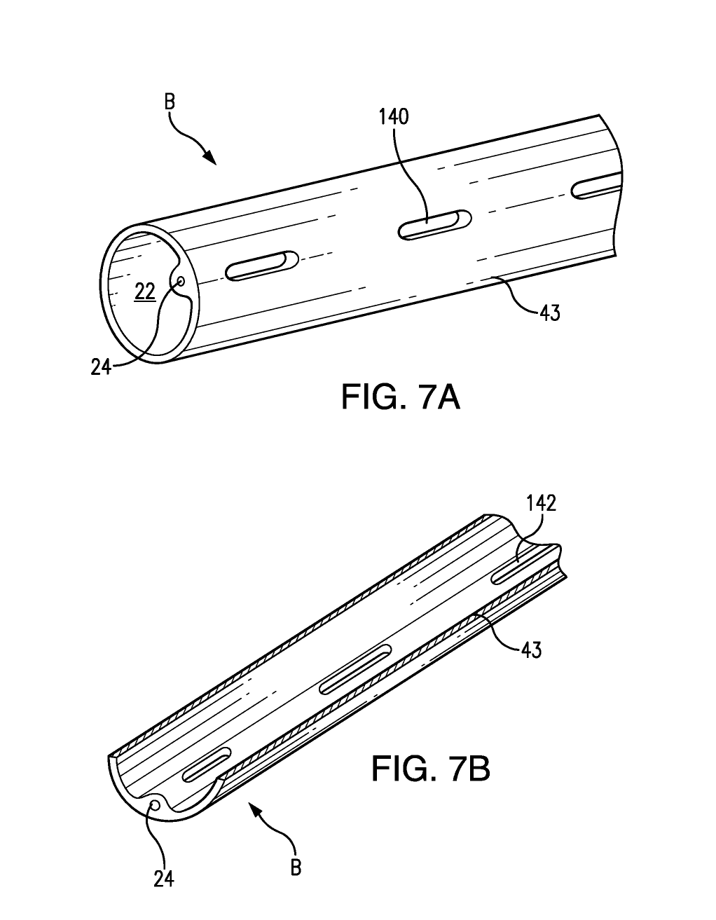

A hemodynamic-concept catheter is shown in FIGS. 7A and 7B. FIG. 7B is a broken partial view of the catheter to illustrate internal features. Conformable section B includes a small lumen, such as stylet lumen 24, within a larger lumen 22. The walls of this smaller lumen 24 contain strategically placed slit-like openings 140, 142 that extend both outward from the catheter (reference numeral 140) and inward to the larger lumen (reference numeral 142). The slit-like openings 140, 142 enable hemodynamic measurements. Insertion of an appropriately shaped style then converts the catheter to a pigtail catheter with additional openings for angiography. In this iteration, the second, smaller, lumen 24 has specifically placed communications to the larger lumen 22 and through the outer wall 43. This allows for transvalvular and sight hemodynamic measurements using only one catheter. A further iteration of the hemodynamic catheter can allow for measurement across the aortic valve.

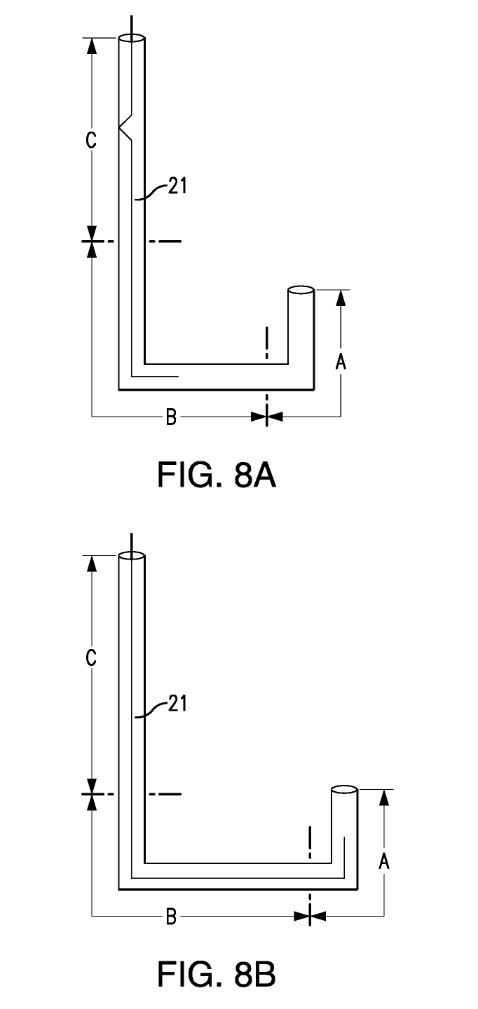

FIGS. 8A-8B illustrate the “Sacky” concept, an in situ transformation from a “Jacky” distal end to a “Sarah” distal end. Jacky and Sarah refer to distal end sizes as disclosed in the Terumo Interventional Systems publication referenced above. By inserting preformed stylet 21 further into conformable lumen section B, it is possible to alter the shape of the catheter from Jacky (FIG. 8A) to Sara (FIG. 8B) some place in between, or for that matter someplace smaller or larger. Removal of the stylet enables a transformation back to Jacky.

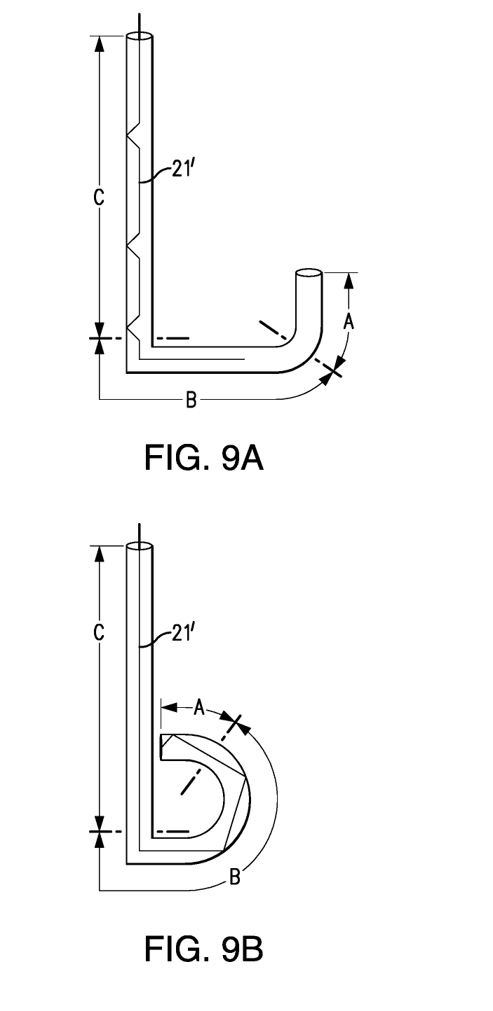

FIGS. 9A and 9B illustrate the use of an alternative stylet 21′ effective to transform a Jacky or Sarah (FIG. 9A) to a pigtail (FIG. 9B).

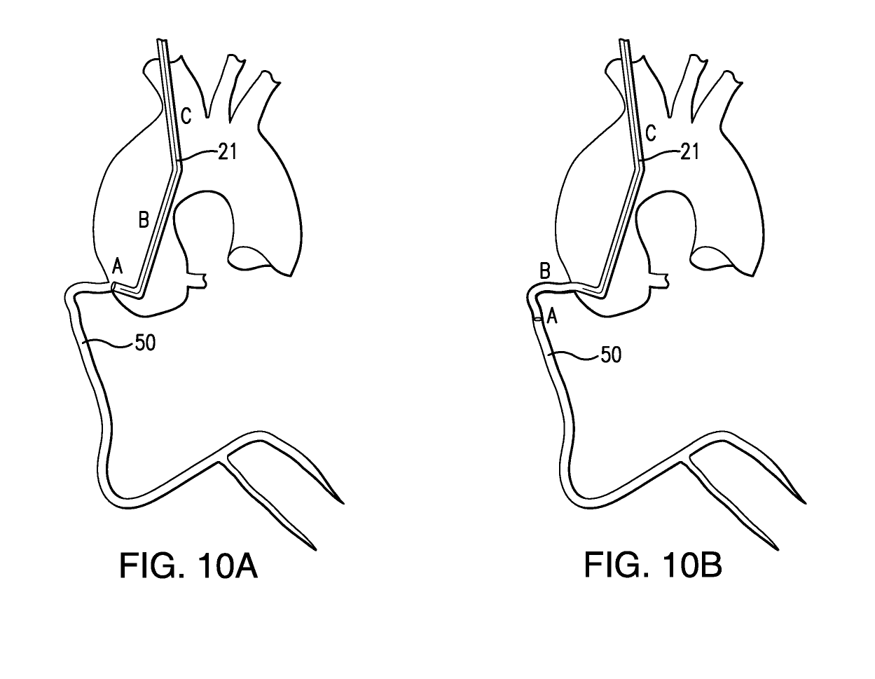

FIGS. 10A and 10 B illustrate the stylets/shaping ribbons disclosed herein as a percutaneous coronary intervention, PCI, guide. The conformable distal end B in its standard alignment is adequate for angiography, but suboptimal for PCI. By extending the tip section A beyond the stylet 21 into a tortuous coronary artery 50, tip section A and conformable distal end B can conform to the vessel 50 and expedite PCI. Alternatively, changing the stylet/guide relative position can also place the guide against the opposing root wall and aid in backup support, again expediting PCI.

Advantages of the catheter systems described herein include angiography can utilize multiple catheters changed in situ if a standard doesn't fit. Because each catheter exchange takes time and introduces risk into the procedure, there is less time and risk involved with in situ changes. There is a reduced need for multiple catheters and reduced requirement for storage space. A need for fewer catheters translates into reduced cost.

Disclosed herein is a shapeable angiography catheter that can be transformed in-situ to an entirely different shape or can be retrofit slightly without a formal catheter exchange. The stylets or shaping ribbons take less storage space and can be modified into a limitless number of shapes for multiple procedures.

One or more embodiments of the present invention have been described. Nevertheless, it will be understood that various modifications may be made without departing from the spirit and scope of the invention. Accordingly, other embodiments are within the scope of the following claims.

1. A catheter assembly, comprising:
a tube having relatively rigid proximal portion bonded to a conformable distal portion with a first lumen extending through both said proximal portion and said distal portion; and
a plurality of stylets each having a different preformed shape and each sized for insertion into said tube, whereby each of said plurality of stylets or shaping ribbons is effective to shape said distal end to a predetermined shape.

2. The catheter assembly of claim 1 wherein said first lumen is effective to introduce an angiography dye.

3. The catheter assembly of claim 2 wherein said first lumen is further effective to receive one of said plurality of stylets.

4. The catheter assembly of claim 2 wherein a wall of said relatively rigid proximal portion and a wall of said conformable portion includes a second lumen that is effective to received one of said plurality of stylets.

5. The catheter assembly of claim 3 wherein a stylet insertion site proximal to said relatively rigid proximal portion is effective for inserting said one of said plurality of stylets into said first lumen.

6. The catheter assembly of claim 4 wherein a stylet insertion site proximal to said relatively rigid proximal portion is effective for inserting said one of said plurality of stylets into said second lumen.

7. The catheter assembly of claim 2 wherein said stylet is effective to transition said conformable portion to a pigtail configuration.

8. The catheter assembly of claim 2 wherein said stylet is effective to transition said conformable portion to a hooked L configuration.

9. The catheter assembly of claim 6 wherein walls of said second lumen include slits to enable fluid communication between said first lumen and an environment exterior to said catheter.

10. A method to use a catheter assembly comprising the steps of:
selecting a catheter having a relatively rigid proximal portion bonded to a conformable distal portion with a first lumen extending through said proximal portion and through said distal portion;
inserting said catheter into a body channel; and
inserting a first stylet having a first preformed shape into said first lumen whereby said distal end assumes a first predefined shape.

11. The method of claim 10 wherein subsequent to inserting said first stylet:
removing said first stylet;
inserting a second stylet having a second preformed shape, that is different from said first preformed shape, into said first lumen whereby said distal end assumes a second predefined shape.

12. The method of claim 11 wherein an angiography dye is introduced into said first lumen prior to removing said first stylet.

13. The method of claim 11 wherein an angiography dye is introduced into said first lumen subsequent to removing said first stylet.

14. A method to use a catheter assembly comprising the steps of:
selecting a catheter having a tubular structure with a relatively rigid proximal portion bonded to a conformable distal portion with a first lumen extending through said proximal portion and through said distal portion, a wall of said tubular structure including a second lumen extending through said proximal portion and through said distal portion;
inserting said catheter into a body channel; and
inserting a first stylet having a first preformed shape into said second lumen whereby said distal end assumes a first predefined shape.

15. The method of claim 14 wherein subsequent to inserting said first stylet:
removing said first stylet;
inserting a second stylet having a second preformed shape, that is different from said first preformed shape, into said second lumen whereby said distal end assumes a second predefined shape.

16. The method of claim 15 wherein an angiography dye is introduced into said first lumen prior to removing said first stylet.

17. The method of claim 16 wherein an angiography dye is introduced into said first lumen subsequent to removing said first stylet.
